(19) United States
(12) Patent Application Publication
(10) Pub. No.: US 2023/0390491 A1
(43) Pub. Date: Dec. 7, 2023

(54) PRESSURE DETECTION SYSTEM AND METHOD

(71) Applicant: CareFusion 303, Inc., San Diego, CA (US)

(72) Inventors: Mohammed Mehtab KHAN, Whitefield Bangalore (IN); Abin AUSTIN, Thrissur (IN); Aman DESAI, Bengaluru (IN); Kanjimpuredathil Muralikrishna MENON, Bangalore (IN); Ryan CALLAHAN, Long Beach, CA (US)

(21) Appl. No.: 18/087,753

(22) Filed: Dec. 22, 2022

Related U.S. Application Data
(60) Provisional application No. 63/349,521, filed on Jun. 6, 2022.
(60) Provisional application No. 63/349,514, filed on Jun. 6, 2022.

(51) Int. Cl. A61M 5/168

(52) U.S. Cl. CPC A61M 5/16831; A61M 2005/16863; A61M 2205/18

(57) ABSTRACT

A pressure detection system includes a body having an internal chamber with input and output ports and a flexible membrane fluidically sealed to an exposed opening of the chamber to prevent a fluid passing through the chamber from passing through the exposed opening. The membrane is configured to change shape responsive to an increase in pressure caused by the fluid within the chamber satisfying a predetermined threshold. In some implementations, the membrane includes or is part of an identification mechanism which moves outwards, away from the chamber, as the pressure increases to indicate the pressure increase. In some implementations, the identification mechanism includes markings on the surface of the membrane, and an image sensing device reads the markings, and provides an indication of a current pressure associated with the fluid in the chamber based on a variation in the markings from a default state.

### CROSS-REFERENCE TO RELATED APPLICATIONS

This application claims the benefit of U.S. Provisional Application No. 63/349,521, filed on Jun. 6, 2022, and U.S. Provisional Application No. 63/349,514, filed on Jun. 6, 2022, the entirety of each of which is incorporated herein by reference for all purposes.

### BACKGROUND

In medical care facilities, infusion of medical fluids into a patient is a commonly performed patient care operation. A fluid infusion device, such as an infusion pump, is typically configured to infuse a fluid from a fluid source into a patient through a vascular access device (VAD) such as a syringe or a catheter. If an occlusion occurs between the pump and the VAD, fluid does not reach the vascular system as intended and blood may back up resulting in clotting and attendant risks.

Prior to starting a fluid delivery session, a caregiver typically sets up the infusion device to alert the caregiver when fluid pressure in the infusion line exceeds a pressure threshold so that the caregiver could take corrective action to avoid possible harm to the patient. Modern infusion devices include built-in pressure sensors for detecting pressure spikes within an infusion line. Current methods of setting up infusion devices include the caregiver setting the pressure limits. In some pumps, pre-configured values may be adjusted by the caregiver while in other pumps the pre-configured values are fixed and all have limited ranges. Certain pumps are pre-configured to acquire a value during power-on, which the caregiver may or may not be allowed to control to adjust, though this acquired value is over a defined range of pressure values.

While existing pressure detection mechanisms have been successful at detecting pressure spikes based on occlusions upstream or downstream of the infusion device, there are no known methods for detecting pressure fluctuations in gravity-based infusion or in the infusion line closer to the patient. It is important to avoid exposure of the patient's vessels and tissue to a higher pressure than necessary and to avoid false alarms which would be issued due to over pressure resulting from an improperly placed gravity feed infusate bag or, for example, from an injection via a Y-valve near the patient. A mechanical syringe, for example, can introduce pressures over 80 psi, placing both the product and the patient at risk of damage or injury.

### SUMMARY

The subject technology provides an inexpensive disposable device that may be placed almost anywhere in an infusion line to detect a alert users of over pressure events by way of providing a visual indication to the user or to a vision-enabled system. The device further provides an overflow that reduces peak pressures.

According to various aspects of the subject technology, a pressure detection system comprises: a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and a flexible membrane fluidically sealed to the exposed opening such as to prevent the fluid from passing through the exposed opening, and configured to change shape and expand responsive to an increase in pressure caused by the fluid accumulated within the chamber; a identification mechanism coupled to the flexible membrane and positioned to move outward, away from the chamber, as the pressure increases and the flexible membrane expands, wherein a distance that the identification mechanism travels responsive to the pressure increase is indicative of the pressure increase.

A method comprises providing a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and fluidically sealing a flexible membrane to the exposed opening such as to prevent the fluid from passing through the exposed opening, and configured to change shape and expand responsive to an increase in pressure caused by the fluid accumulated within the chamber; coupling an identification mechanism to the flexible membrane and positioned to move outward, away from the chamber, as the pressure increases and the flexible membrane expands, wherein a distance that the identification mechanism travels responsive to the pressure increase is indicative of the pressure increase.

According to various aspects of the subject technology, a pressure detection system comprises a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and a flexible membrane fluidically sealed to the exposed opening such as to prevent the fluid from passing through the exposed opening, and configured to change shape responsive to a pressure caused by the fluid accumulated within the chamber satisfying a predetermined threshold, the flexible membrane comprising one or more markings on a surface of the flexible membrane that deform when the flexible membrane changes shape.

In some implementations, the pressure detection system further comprises an image sensing device; and one or more processors configured to: cause the image sensing device to read the one or more markings on the surface of the flexible membrane; measure, based on the image sensing device reading the one or more markings, a current variation from a default state in the one or more markings; and provide an indication of a current pressure associated with the fluid in the chamber based on the current variation. Other aspects include corresponding methods, apparatus, and computer program products for implementation of the corresponding system and its features.

According to various aspects of the subject technology, a method comprises providing a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and fluidically sealing a flexible membrane to the exposed opening such as to prevent the fluid from passing through the exposed opening, and configured to change shape responsive to a pressure caused by the fluid accumulated within the chamber satisfying a predetermined threshold, the flexible membrane comprising one or more markings on a surface of the flexible membrane that deform when the flexible membrane changes shape.

In some implementations, the method further comprises configuring an image sensing device to read the one or more markings on the surface of the flexible membrane; configuring a processor to measure, based on the image sensing device reading the one or more markings, a current variation from a default state in the one or more markings; and configuring the processor to provide an indication of a current pressure associated with the fluid in the chamber based on the current variation. In some implementations, the method further comprises configuring the processor to determine that the current variation in the one or more markings corresponds to the current pressure satisfying a predetermined threshold pressure; and configuring the processor to provide a notification regarding the current pressure satisfying the predetermined pressure threshold. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example gravity-based fluid delivery system, according to various implementations of the subject technology.

FIG. 2 depicts an example pump-driven fluid delivery system, including an infusion pump shown in use in its intended environment, according to various aspects of the subject technology.

FIGS. 3A through 3D depict a first example pressure detection apparatus, according to various aspects of the subject technology.

FIGS. 4A through 4D depict a second example pressure detection apparatus, according to various aspects of the subject technology.

FIG. 5 depicts an example pressure detection system configured for use in connection with the second example pressure detection apparatus, according to various aspects of the subject technology.

FIGS. 6A through 6E depict a third example pressure detection apparatus, according to various aspects of the subject technology.

FIGS. 7A through 7C depict a fourth example pressure detection apparatus, according to various aspects of the subject technology.

FIG. 8 depicts a fifth example pressure detection apparatus, according to various aspects of the subject technology.

FIG. 9 depicts a first example process for fabricating or otherwise providing a pressure detection apparatus, according to various aspects of the subject technology.

FIG. 10 depicts an example process for detecting a pressure fault with a pressure detection system, according to various aspects of the subject technology.

FIG. 11 depicts a second example process for fabricating or otherwise providing a pressure detection apparatus, according to various aspects of the subject technology.

FIG. 12 is a conceptual diagram illustrating an example electronic system for facilitating pressure sensing in a pressure detection system, according to aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The disclosed pressure detection and pressure notification system includes a single use disposable unit which includes a fluid chamber sealed with a flexible membrane and which is placed in a fluid path of an infusion. The unit may be attached to a capital equipment setup such as an image processing unit in communication with an infusion device and/or notification server. Based on pressure in the system, the flexible membrane on the device expands or collapses, and the membrane may include a printed pattern that changes design with the expansion or collapsing of the membrane. The pattern may be detected by the image processing unit to alert a clinician of pressure fluctuations or to control an infusion pump (e.g., by automatically terminating the infusion responsive to an over pressure).

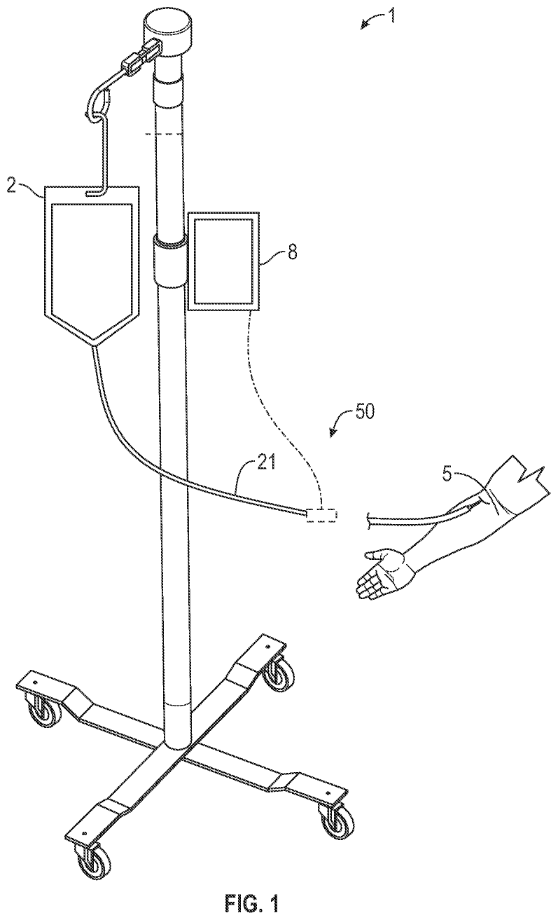

FIG. 1 depicts an example gravity-based fluid delivery system, according to various implementations of the subject technology. The example delivery system includes a fluid container 2 containing an intravenous (IV) fluid is held on an intravenous (IV) pole. According to various implementations, the fluid source is a malleable fluid container such as an IV bag or blood product bag. An infusion line 21 is connected to the malleable fluid container 2 for delivery of the fluid to the patient. The infusion line 21 may be a conventional IV infusion-type tube typically used in a hospital or medical environment, and is made of any type of flexible tubing appropriate for use to infuse therapeutic fluids into a patient, such as polyvinylchloride (PVC). A cannula 5 is mounted at the distal end of the flexible IV tubing for insertion into a patient's blood vessel or other body location 22 to deliver the fluid to the patient.

Administration of IV fluids, regardless of the container, requires that a fluid container 2 be suspended by a at some height, typically 0.5-1.0 meter, above the patient or an infusion pump. The container 2 is then connected by a flexible tube 21 to either the patient directly or to an infusion pump. The administration may include a drip chamber (not shown). Relatively inexpensive tubing may be used such as polyvinyl chloride (“PVC”) tubing or similar type tubing.

Flow may be achieved by either gravity-pressure or positive-pressure. Gravity-pressure based flow control systems rely on the force of gravity for fluid flow. In this regard, mounting the fluid container above the delivery point generates a positive pressure due to gravity at the connection of the infusion tube to the patient or pump.

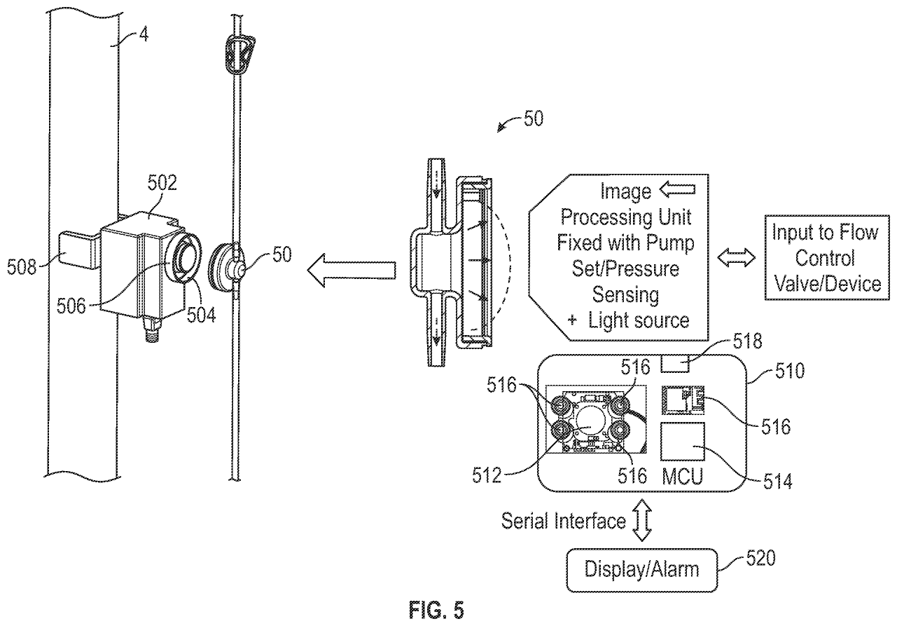

Some systems may include an “IV controller” which interfaces with the IV tube. An IV controller is a device that automatically controls the flow rate of fluid through the IV tube by use of a pinching device that pinches the tube more or less to control the flow of fluid therethrough. An IV controller may be responsive to a control signal generated by, for example, a flow sensor attached to the drip chamber. The flow sensor senses fluid drops falling in the drip chamber, and a flow rate calculated based on counting the number of drops per unit time. If the calculated flow rate is greater than a desired flow rate, the controller adjusts the pinching device to lower the flow rate by pinching the tube further. Advantages of gravity administration sets include their relative simplicity and low cost. As will be described further, a pressure detection device 50 may be included for sensing and/or relieving pressure within the infusion line 21. In some implementations, a computing device 8 and/or display, may be attached to the IV pole to facilitate readings from the pressure detection device. For example, as shown in FIG. 5, an image sensing device may be configured to read pressure from the pressure detection device and transmit the pressure information to the computing device 8 for interpretation by a user.

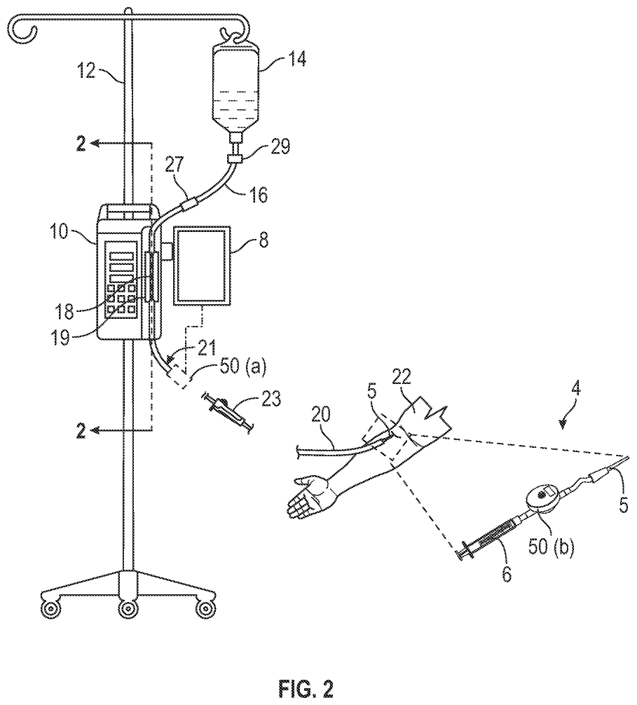

FIG. 2 depicts an example pump-driven fluid delivery system, including an infusion pump 10 shown in use in its intended environment, according to various aspects of the subject technology. In the depicted example, a fluid source 2 is connected in fluid communication with an upstream portion 16 of fluid line 21. A flexible portion 18 of the fluid line is mounted in operative engagement with a peristaltic pumping apparatus 19 for propelling fluid through a downstream fluid line 20, for example, to a patient's arm 22. A roller clamp 23 (e.g., configured to provide for mechanical compression of the line to block the flow) may be positioned on the downstream fluid line 20 between the infusion pump 10 and the patient's arm 22 via a cannula 5.

In some implementations, the pressure detection device 50 (a) may be connected in the infusion line 21, downstream of the pump 10 (e.g., above or below the roller clamp 23). In some implementations, the pressure detection device 50 (b) may be coupled to an infusion extension set 4 and/or inserted between a syringe 6 and the catheter 5. In the depicted example (b), the pressure detection device 50 may be used to monitor pressure of an infusion provided via the extension set 4 by the syringe 6.

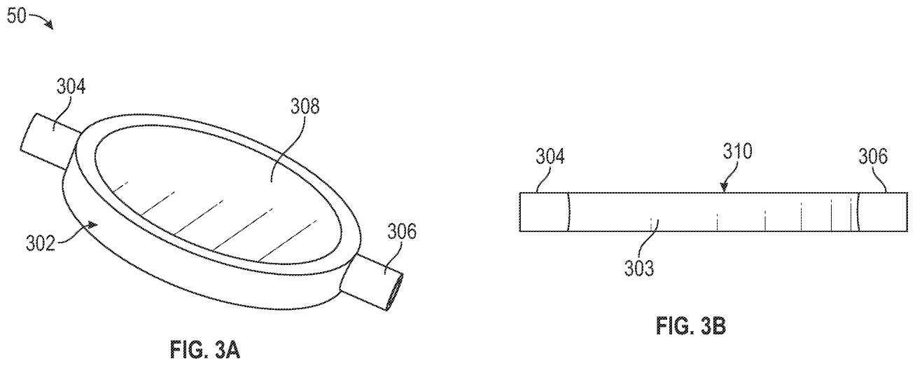

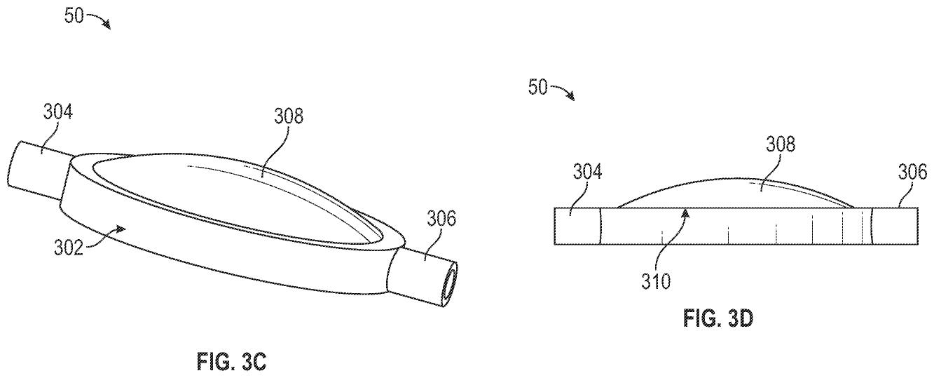

FIGS. 3A through 3D depict a first example pressure detection apparatus, according to various aspects of the subject technology. As depicted, the subject technology may include a component made of a rigid plastic body and flexible membrane that can be inserted into a fluid path such as an infusion line. As pressure in the system increases the flexible membrane, which stretches to relieve pressure in the system. When the source of pressure increase is removed the membrane returns to its normal state. For the purpose of this disclosure, the flexible membrane may be a material that is responsive with respect to a pressure change or force upon it. Suitable materials may be from the elastomeric family. The material may regain or retract to its original shape when the net pressure on it reduces to zero or becomes minimal or negligible.

The depicted examples illustrate a pressure detection system 50 that includes a body 302 with a substantially hollow chamber 303 disposed between an input port 304 an output port 306. The chamber 303 is configured to accumulate fluid from an upstream portion of an infusion line fluidly coupled to the input port 304, and to supply the fluid to a downstream portion of the infusion line fluidly connected to the output port 306. According to various implementations, the chamber 303 may be in the form of a basin with the input and output ports fluidically formed with sides of the basin. A flexible membrane 308 covers the basin opening 310 and is fluidically sealed to the opening of the basin such as to prevent the fluid from flowing out of or passing through the exposed opening.

The flexible membrane 308 is configured to change shape responsive to a pressure caused by the fluid accumulated within the chamber. As depicted, the flexible membrane is configured to project away from chamber responsive to the pressure becoming greater than a pressure threshold. In this regard, according to various implementations, the flexible membrane 308 may be of a predetermined thickness and shape configured to flex and deform responsive to the pressure caused by the fluid accumulated within the chamber 302. In some implementations, the flexible membrane may be flexible such that it stretches and expands responsive to pressure building within the chamber 303. The flexible membrane may be flat with the basin opening 310 as depicted in FIG. 3B, and then begin to expand, for example, beyond the plane of the basin opening 310 when the pressure satisfies a predetermined pressure threshold, as depicted in FIGS. 3C and 3D. In some implementations, the basin opening is circular, and the flexible membrane 308 expands according to a convex shape, as depicted in FIGS. 3C and 3D.

In some implementations, the flexible membrane may flex and form to one or more predetermined states of curvature, depending on the pressure within the chamber 302. In some implementations, the flexible membrane is configured to be flat until the pressure within the chamber satisfies a pressure threshold, and then take on a predetermined convex shape when a pressure within the chamber satisfies the threshold. In this regard, the flexible membrane may be flat when in a default or normal state while at a first predetermined pressure threshold, and then take on a convex shape when in the expanded state responsive to the pressure satisfying a second predetermined pressure threshold.

According to some implementations, the chamber 303 and flexible membrane 308 are configured to operate together to reduce a pressure buildup within the chamber 303 when switched to the expanded shape. The membrane may flex and/or expand to allow pressure relief within the fluid system. The material and stiffness of flexible membrane may be adjusted to change pressure required to activate relief action. Size of device may also be adjusted to change total volume of fluid that is contained in activated state. In this regard, the device operates as a pressure relief valve and provides an overflow that reduces the peak pressure generated, while also providing a visual indication (e.g., by way of the expanded shape) to the user than an over pressure event has occurred.

Some implementations, the flexible membrane 308 may expand in a tamper-proof manner whereby the flexible membrane is prevented from returning to the default shape after expanding to the expanded shape. For example, the flexible membrane 308 may be made of a material that stretches but does not retract to its original shape, or may be of a synthetic or semisynthetic material (e.g., polymer based material) that switches from a default shape (e.g., flat or concaved) to the depicted convex shape of FIGS. 3C and 3D. In some implementations, as will be described further, the membrane 308 may be printed with markings that indicate how much pressure has been generated.

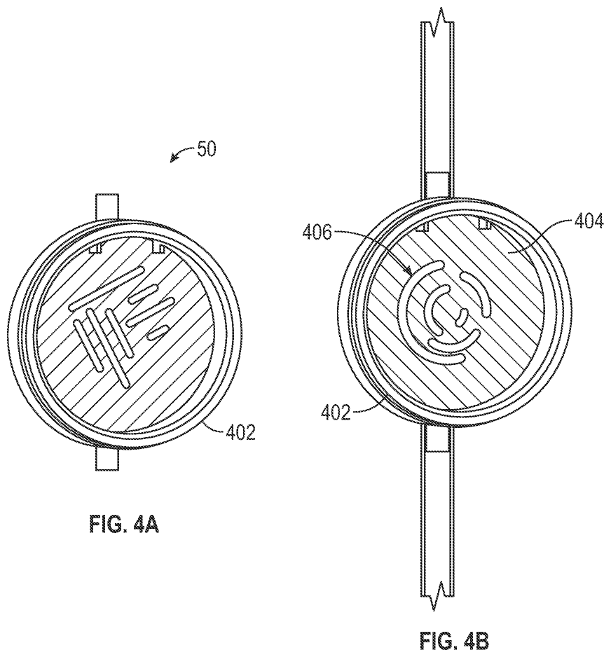

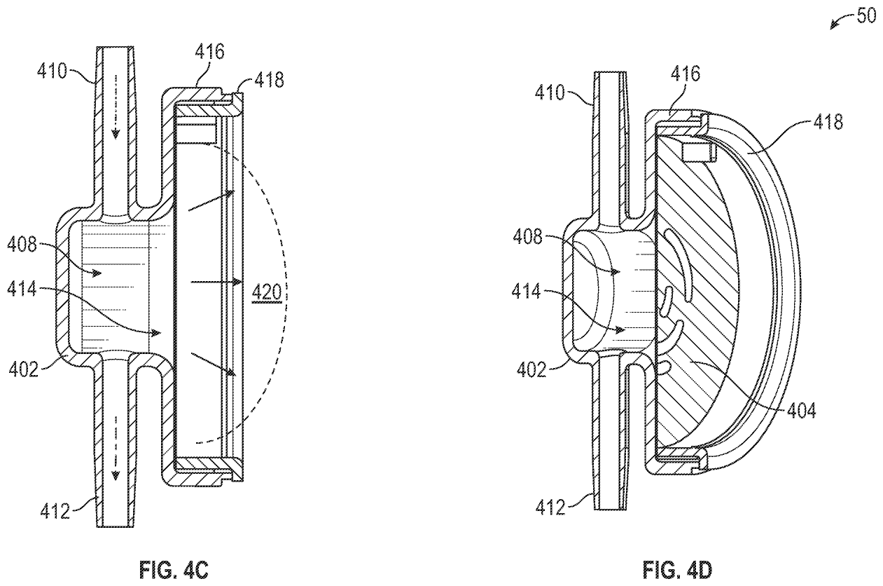

FIGS. 4A through 4D depict a second example pressure detection apparatus 50, according to various aspects of the subject technology. As depicted, the subject system may include a component made of a rigid plastic body 402 and flexible membrane 404 that can be inserted into a fluid path such as an infusion line. As pressure in the system increases the flexible membrane 404, which stretches to relieve pressure in the system. The flexible membrane 404 may be encased in the plastic body 402 and coated and/or printed with one or more markings 406 that facilitate identification of a pressure within the device when the membrane 404 is in an expanded state.

According to the depicted implementation, the disclosed device may include a chamber 408 comprising an input port 410 and an output port 412, and configured to accumulate fluid from an upstream portion of an infusion line fluidly coupled to the input port 410, and to supply the fluid to a downstream portion of the infusion line fluidly connected to the output port 412. A flexible membrane 404 is fluidically sealed to an exposed opening 414 of the chamber 408 such as to prevent the fluid from passing through the exposed opening 414. According to various implementations, the flexible membrane 404 is configured to expand and change shape responsive to a pressure caused by the fluid accumulated within the chamber 408.

As shown in FIGS. 4A through 4D, the chamber may expand into a housing 416 having a larger diameter than the chamber, such that the floor of the housing circumscribes the chamber opening and the wall(s) and floor of the housing forms a well within which the flexible membrane 404 is positioned. As depicted, the flexible membrane 404 may be fluidly sealed by way of a frame 418 that traverses along and/or conforms to an inner side of the wall(s) of the well. The frame may sandwich and constrain the membrane 404 between the floor and the frame and/or between the wall(s) and/or the frame. In this manner, the flexible membrane 404 may expand 420 within the well, as depicted in FIG. 4C.

According to various implementations, the markings 406 on the surface of the flexible membrane 404 deform when the flexible membrane 404 changes shape. For example, as depicted in FIG. 4A, the markings may include one or more lines printed on the membrane in a manner such that, when the flexible membrane is in the default state (e.g., flat) the markings are straight, and when the membrane is in the expanded state (e.g., convex) the markings are deformed (e.g., curved). In this regard, an indication that the markings 406 have deformed may indicate a pressure change in the chamber. In some implementations, as depicted in FIGS. 4B and 4D, the markings may deform into a pattern of curved lines according to an amount of curvature of the flexible membrane (e.g., when in the convex shape). In this manner, the magnitude of the deformation or the pattern formed by the curvature of the membrane as it expands may be associated with a pressure value. For example, the pattern may be compared against predetermined patterns that are each associated with a pressure value, and the current pressure within the chamber determined based on indexing the pressure by the depicted pattern.

FIG. 5 depicts an example pressure detection system configured for use in connection with the second example pressure detection apparatus, according to various aspects of the subject technology. The depicted system includes the pressure detection apparatus 50 in combination with an image sensing device 502. The image sensing device 502 includes image sensing instrument 504 such as a camera capable of capturing images for subsequent vision processing by a processor. The image sensing device 502 is located and positioned so that the image sensing instrument 504 of the device 502 is aligned with the markings on the flexible membrane of the pressure detection apparatus 50.

In some implementations, the image sensing device 502 includes a coupling mechanism 506 that couples to the housing of pressure detection apparatus 50 and aligns the image sensing instrument 504 with the markings. In the depicted example, the coupling mechanism 506 is a circular rim that snaps together with the housing of the pressure detection apparatus, thereby centering the markings in the center of the membrane with one or more lenses of the image sensing device centered within the circular rim. For convenience, the image sensing device 502 may include a pole attachment 508 for coupling the device 502 to an IV pole 4. In this regard, the system 50 may position the apparatus 50 such that the input port and output port (and infusion line 21) are vertically aligned. In this manner, the gravitational forces operating on a fluid are more likely to remain predictable, and pressure induced expansion of the flexible membrane 404 and resulting patterns generated thereby are more precisely correlated with predetermined expected pressure values.

The image sensing device 502 may include or be connected to one or more processors. In the depicted example, device 502 includes an internal circuit board 510 with a miniature camera 512 and a microprocessor 514 operating the camera 510 for sensing the markings 406 of the flexible membrane 404. An LED array 516 may be included to illuminate the markings for the camera 512. The internal circuit board 510 may further include a wireless circuit 516 (e.g., Bluetooth or RF communication circuit), or a wired interface 518 (e.g., a USB communication interface), for communication with a remote computer system or an operably coupled infusion device 10. In some implementations, a serial interface 520 may be included for interfacing with external devices, such as an external display or alarm. In some implementations, an external computing device (e.g., a mobile device) may operably connect to the device 502 via the wireless interface or wired interface and control operation of, or collect data from, the device. For example, the sensing device 502 and/or an operable connected server connected to the device 502 may notify a clinician about a pressure surge in an infusion line via the clinician's mobile phone or device (via Bluetooth or Internet connection). In some implementations, various operations of an infusion device may be triggered by signals provided by the device 502 responsive to sensing pressures in the apparatus 50.

According to various implementations, the processor 514 of the device 502 (or, e.g., a remotely connected processor) may be programmed to cause the image sensing device 502 to read the marking(s) on the surface of the flexible membrane 404, and then based on the markings 406 measure a current variation of the membrane 404 from a default state. As described previously, the markings 406 may include a plurality of straight lines when the flexible membrane 404 is flat. These lines may then deform into a pattern of curved lines according to an amount of curvature of the flexible membrane when in the convex shape. The processor is programmed to detect and match the pattern of curved lines with one or more predetermined patterns, and determine the current pressure based on indexing a matched pattern with a predetermined pressure value.

According to some implementations, the processor(s) may be further programmed to determine an expansion state of the flexible membrane based on the markings read from the surface of the flexible membrane. For example, the expansion state may include an amount of shape change of the membrane from a default state. The processor(s) may be further programmed to determine a deviation in the pressure within the chamber from a baseline pressure based on the determined expansion state.

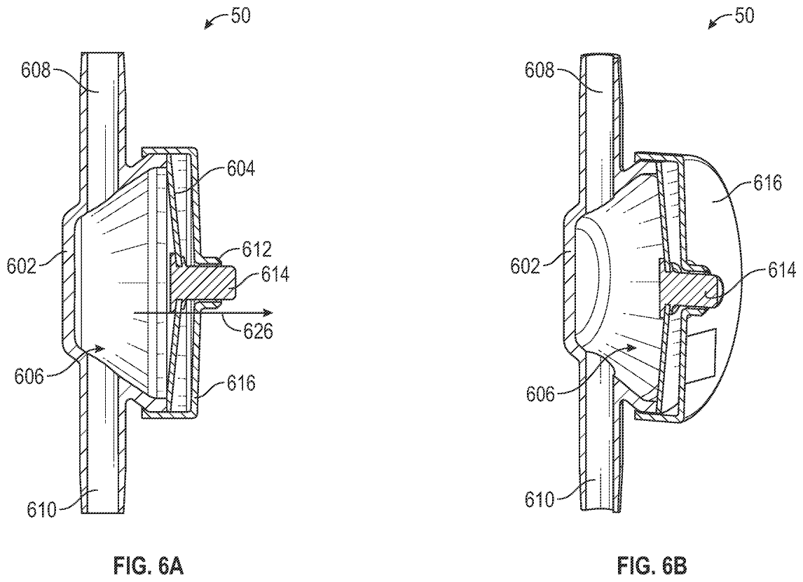

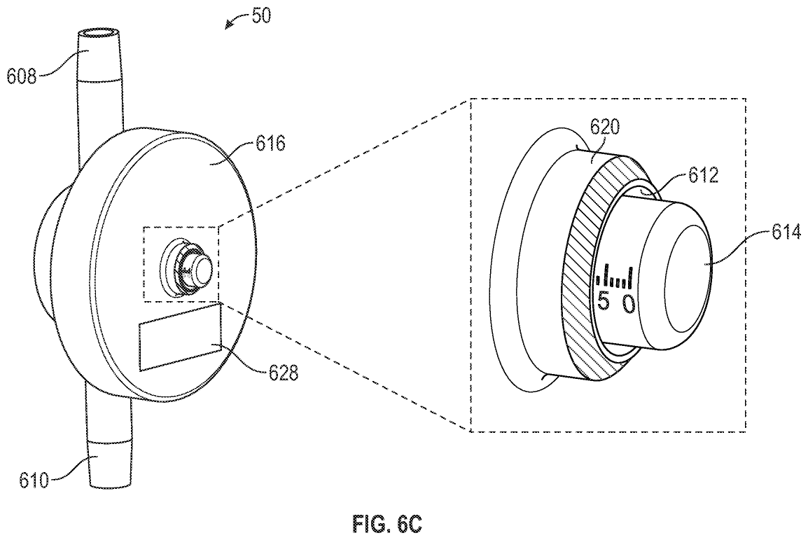

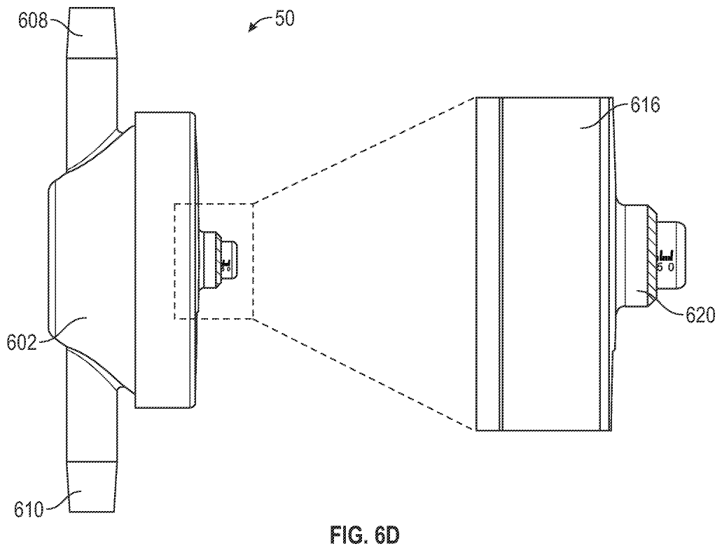

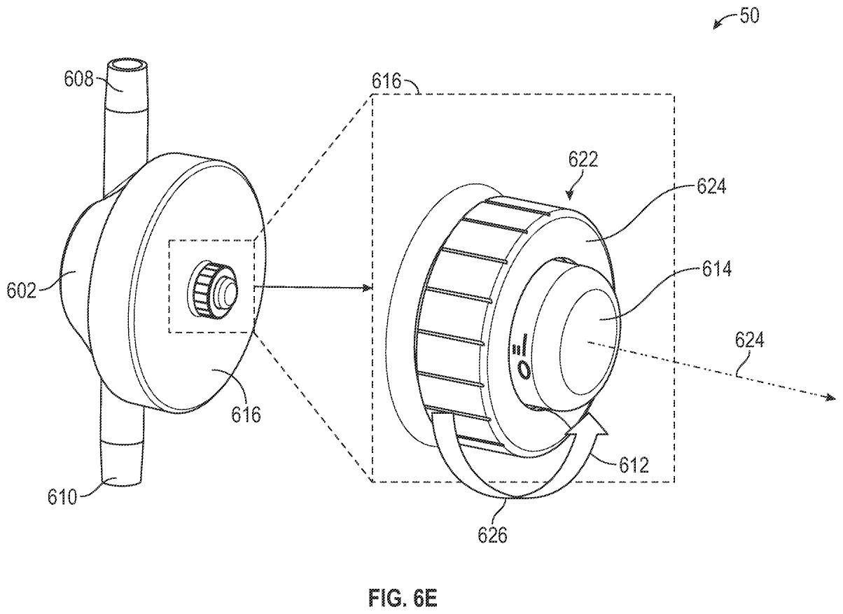

FIGS. 6A through 6E depict a third example pressure detection apparatus 50, according to various aspects of the subject technology. As depicted, the subject system may include a component made of a rigid body 602 and flexible membrane 604. As previously described, the body 602 includes an internal chamber 606, an input port 608 and an output port 610. The chamber is configured to accumulate fluid from an upstream portion of an infusion line 21 fluidly coupled to the input port 608, and to supply the fluid to a downstream portion of the infusion line 21 fluidly connected to the output port 610. The flexible membrane 604 is fluidically sealed to an exposed opening 612 of the chamber 606 such as to prevent the fluid with the chamber 606 from passing through opening 612. As previously described, the flexible membrane 604 is made from a material (e.g., elastomeric material) configured to change shape and expand responsive to an increase in pressure caused by the fluid accumulated within the chamber 606.

In the depicted implementations, the pressure detection apparatus 50 includes an identification mechanism 614 coupled to the flexible membrane 604. According to various implementations, the identification mechanism 614 includes a plunger positioned to project outward, away from the body 602 and chamber 606. According to various implementations described herein, the plunger 614 may include or be in the form of a cylinder, rectangular, or other oblong appendage coupled to the surface of the membrane. A casing 616 encompasses at least a portion of the flexible membrane and is coupled to at least a portion of the body 602. The casing 616 includes an aperture 618 at a location of the plunger 614, the plunger passing through the aperture 618, as depicted. In some implementations, the aperture may include a collar 620 surrounding a portion of the plunger 614.

In this regard, as the pressure increases within the chamber 606 and the flexible membrane 604 expands, the identification mechanism 614 is positioned to move outward 626, away from the chamber 606. As the pressure increases and the flexible membrane expands, the plunger moves to extend further beyond the aperture and the casing (and the collar). The distance that the plunger/identification mechanism travels responsive to the pressure increase is indicative of the pressure increase.

The plunger 614 may include markings or indicators (e.g., physical etchings) to indicate a pressure reading. In the depicted example, the plunger 614 includes graduated values, which may be referenced with regard to the collar 620 and/or protective casing 616. In this regard, the edge of the collar/casing may identify a value/location on the plunger 614 while in a pressurized state. In this manner, as the IV line 21 is primed, a value indicative of the initial static pressure (in the chamber) may be read from graduated readings on the plunger 614. The clinician may mark the plunger at this value. In some implementations, a surface of the casing 626 may be suited for marking, and an area 628 on the surface may be provided for marking the initial value of the plunger 614. When the catheter starts to occlude, static pressure builds up and causes the flexible membrane 604 to bulge and the graduated plunger moves to reveal a new value with respect to the collar/casing. This new value may then be compared to the previous value to determine whether the line has occluded and the extent of the occlusion.

As depicted in FIG. 6E, the pressure detection apparatus 50 may include a taring mechanism 622 operatively coupled to the casing 616 and mechanically movable in a lateral direction 624 along an axis and/or length of the plunger 614 with respect to the casing 616. In this regard, a portion of the plunger—e.g., a top rim 624 of the plunger—may be used to identify a location on the plunger.

In some implementations, the casing 616 includes a threaded collar 620 (not shown), which includes the aperture 612. In this regard, the taring mechanism 622 may be coupled to the casing 616 by way of being threaded onto threads of the threaded collar 620. The taring mechanism 622 may be mechanically movable by way of being turned about 626 the collar 620 according to the threads of the threaded collar 620.

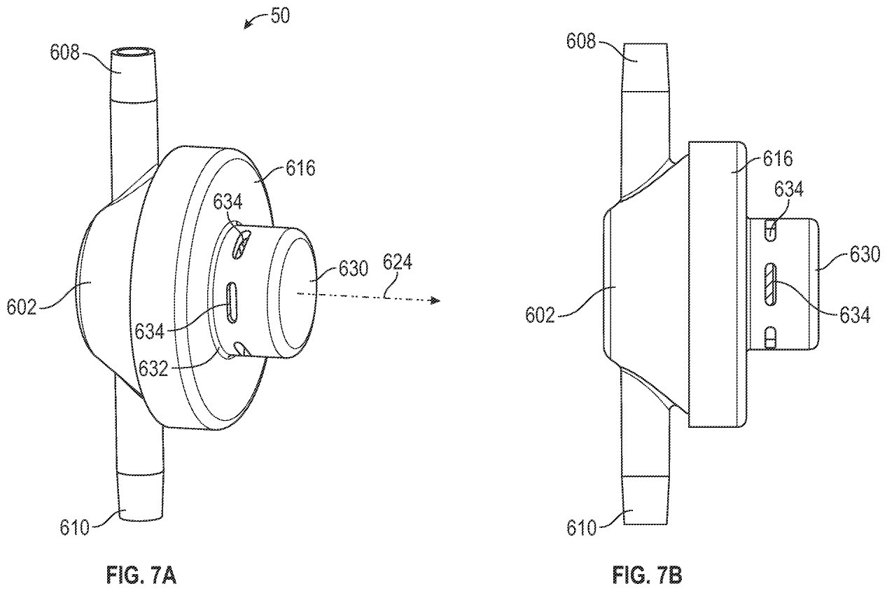

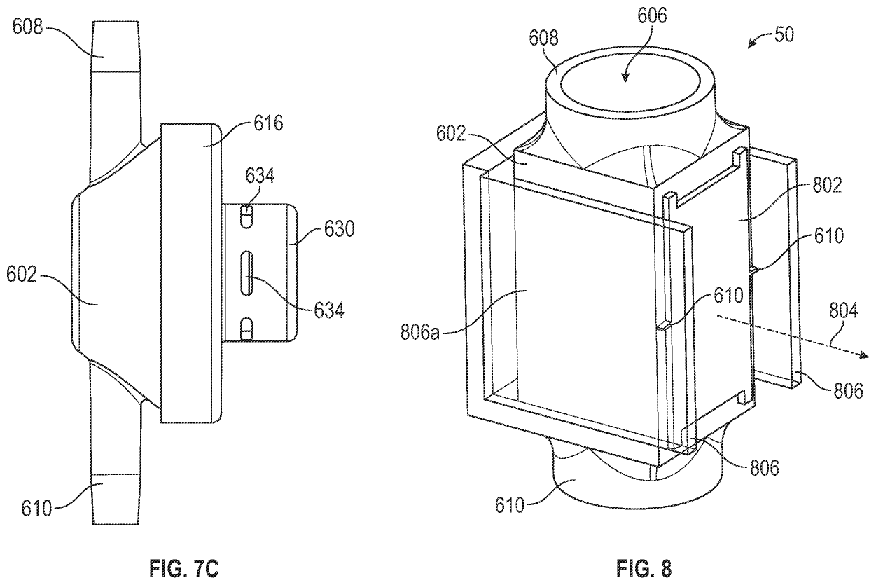

FIGS. 7A through 7C depict a fourth example pressure detection apparatus 50, according to various aspects of the subject technology. The depicted implementation is similar to the implementations of FIGS. 6A through 6E, with additional features. The depicted implementation includes a plunger housing 630 coupled to an outer portion 632 of the casing 616 such as to encompass the plunger 614 and the aperture 612. In this regard, the housing 630 may replace the collar 620 and/or taring mechanism 622 of FIG. 6. The housing 630 may be a separate component from the casing 616, or may be formed as part of the casing 616 (e.g., the casing and housing may be a single component).

In the depicted implementation, the plunger 614 is entirely enclosed within the casing and housing. The housing 630 includes an opening 634 which exposes a portion of the plunger 614 within the housing to a user who is viewing the device 50. The plunger may then include one or more identifiers laterally disposed on the plunger that can be read through the opening to identify a pressure value. A first identifier and a second identifier may be laterally disposed on the plunger, and the first identifier (e.g., green) may be exposed through the opening 634 when the plunger is in a first position associated with a first pressure, and the second identifier may be exposed through the opening when the plunger is in a second position associated with a second pressure.

In the depicted example, the plunger is color coded, with a first area of the plunger 614 associated with a safe pressure color coded green, and a second area of the plunger not associated with the safe pressure color coded red. For example, the opening may be positioned proximate the casing (closer to the flexible membrane) and a lower portion of the plunger, closer to the casing and/or membrane may be colored green so that when the flexible membrane is in a default position (e.g., nearly flat), the portion of the plunger colored green is viewable through the opening. Other portions of the plunger (e.g., farther from the membrane and/or casing) may be colored red so that when the flexible membrane is in a position associated with a higher pressure (e.g., expanded), the portion of the plunger colored red is viewable through the opening to alert/warn the clinician of a possible pressure fault such as an occlusion.

In some implementations, similar to the implementation of FIG. 6E, the plunger housing 630 is operatively coupled to the casing and mechanically movable in a lateral direction 624 along the length of the plunger with respect to the casing such as to reposition the opening. For example, the housing 630 may be threaded at the end closes to the portion 632 of the casing 616, which may include a threaded aperture that receives the threaded portion of the housing. In this regard, the housing 630 may be rotated in either direction to laterally move the opening 634. In this regard, when the pressure is stable (e.g., during a priming operation), the opening may be repositioned so that the green portion (or other first identifier) of the plunger is visible through the opening (FIG. 7B). When a pressure fault occurs, the plunger moves such that the red portion (or other second identifier) of the plunger is visible through the opening (FIG. 7C), thereby indicating the pressure fault.

FIG. 8 depicts a fifth example pressure detection apparatus 50, according to various aspects of the subject technology. The depicted implementation is similar to the implementations of FIGS. 1 through 7, with additional features. As described previously, a body 602 includes an internal chamber 606, an input port 608 and an output port 610. The chamber is configured to accumulate fluid from an upstream portion of an infusion line 21 fluidly coupled to the input port 608, and to supply the fluid to a downstream portion of the infusion line 21 fluidly connected to the output port 610. A flexible membrane (not shown in FIG. 8) is fluidically sealed to an exposed opening (not shown) of the chamber 606 such as to prevent the fluid with the chamber 606 from passing through the opening.

In the depicted example, the flexible membrane prevents the fluid within the chamber from escaping by working in conjunction with the plate 802, which is coupled to the membrane. Instead of the flexible membrane forming a diaphragm (e.g., in FIGS. 3-7), the flexible membrane, together with the plate, forms a bellow at the chamber opening (e.g., with the membrane clamped within the body). The membrane sits within the chamber opening and expands out as pressure increases. The plate 802 is coupled to the membrane and positioned parallel to the chamber, as depicted. The plate 802 may sit flat on the surface of the body.

The membrane may be made of an elastomeric material bound at all four edges of the chamber, internally within the chamber. According to various implementations, the flexible membrane is fluidically sealed to an inner side of an interior of the chamber and, as the pressure increases the flexible membrane expands and the plate moves unidirectionally away 804 (e.g., all points travel together at the same time) from the body and the chamber. The membrane may not be bound a certain distance within the chamber from the edge to allow retraction within the chamber, while allowing stretching beyond the chamber. According to FIG. 8, the plate 802 (aka valve or bellow) moves horizontally/laterally when there is a static pressure acting on it.

In some implementations, the flexible membrane is covers the opening of the chamber, as described previously, and the plate 802 is fastened (e.g., glued) to a substantial portion of the flexible membrane. In some implementations, the flexible membrane and plate together form a four sided bellow with each of the four sides at least partially disposed within the chamber. In the depicted example, the plate 802 is rectangular with flat edges; however, the plate may be other shapes such as an ovoid or circular.

As depicted, the body 602 may be a rectangular structure with a squared or rectangular cross-section, such that the body 602 includes flattened sides. One side may include the flattened plate 802, while the other sides may also be substantially flattened. In some implementations, the pressure detection apparatus 50 includes one or more transparent panels 806 coupled to one or more sides of the body. Each panel 806a and 806b adjacent to the plate 802 may be parallel to a flat side of the body 602 and perpendicular to the plate 802. A transparent panel 806 on a side of the body and perpendicular to the plate 802 may extend beyond the plate on the side of the body beyond which the plate moves responsive to a pressure increase. In this regard, the plate 802 may be viewed through the transparent panel 806 as it moves due to pressure with the chamber.

The plate 802 may include an indicator 1208 that is viewable through the panel 806. A panel 806a may include a transparent scale that corresponds to the indicator on the panel 802. The movement of an edge of the plate from the body, and distance that the plate travels responsive to the pressure increase, is viewable through the transparent panel. Accordingly, as the panel moves 804, the indicator may be tracked according to the scale. When the pressure is in a steady state (e.g., during a priming operation), a clinician may mark the initial reading on the scale. When there is an occlusion in the line 21 or a cause for rise in static pressure, the plate moves outwards 804. As the plate moves outwards 804, the clinician may note down the relative change in the static pressure with the help of the transparent scale. The indicator 1208 on the plate 802 aids in the reading of the pressure change from the scale. The indicator may be a marking on the edge of the panel or, in some implementations, may be a protrusion (e.g., similar to a fingertip).

In some implementations, one or more of the panels may be removably coupled. In this regard, a clinician may mark the panel with a pen at a current position of the indictor, and the panel 806a may be unmounted from the body 602 and measured at a later time to view the range of movement.

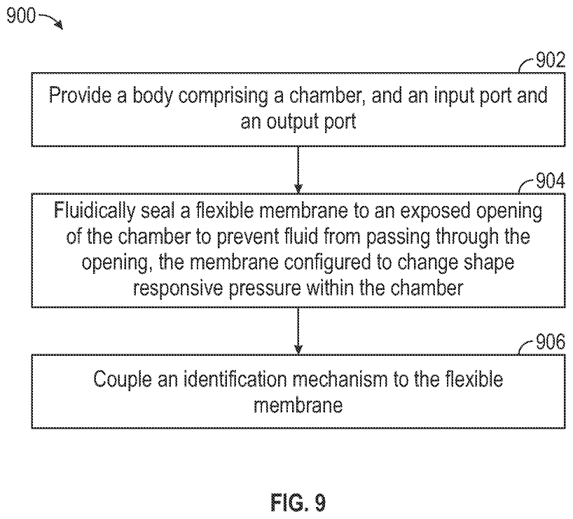

FIG. 9 depicts a first example process for fabricating or otherwise providing a pressure detection system, according to various aspects of the subject technology. For explanatory purposes, the various blocks of example process 900 are described herein with reference to FIGS. 1-8, as well as the components and processes described herein. In some implementations, one or more of the blocks may be implemented apart from other blocks, and by one or more different processors or devices. Further, for explanatory purposes, the blocks of example process 900 are described as occurring in serial, or linearly. However, multiple blocks of example process 900 may occur in parallel. In addition, the blocks of example process 900 need not be performed in the order shown and one or more of the blocks of example process 900 need not be performed.

In the depicted example, a body (e.g., body 302, 402, 602) comprising an internal chamber (e.g., chamber 303, 408 606), and an input port (e.g., port 304, 410, 608) and an output port (e.g., port 306, 412, 610) is provided (902). The body chamber is configured to accumulate fluid from an upstream portion of an infusion line fluidly coupled to the input port, and to supply the fluid to a downstream portion of the infusion line fluidly connected to the output port.

A flexible membrane (e.g., membrane 308, 404, 604) is fluidically sealed to an exposed opening of the chamber (e.g., causing it to no longer be exposed) such as to prevent the fluid from passing through the exposed opening (904). According to various implementations, the flexible membrane is configured to change shape responsive to a pressure caused by the fluid accumulated within the chamber. In the examples of FIGS. 3, 4, and 6, the flexible membrane functions as a dome over the exposed opening.

In some implementations, the change in shape of the membrane is gradual and/or proportional to the pressure. In some implementations, the change occurs responsive to the pressure satisfying a predetermined threshold. The flexible membrane may be configured to be flat when the current pressure satisfies a first predetermined pressure threshold, and to take on a convex shape responsive to the current pressure satisfying a second predetermined pressure threshold. For example, when the fluid in the chamber is at a normal flow pressure for the infusion line (e.g., under normal atmospheric conditions), the membrane may remain flat. Upon a negative pressure being introduced, the membrane may become concaved into the chamber. Upon a positive pressure, the membrane may become convex, ballooning out away from the chamber.

In the example of FIG. 4, the flexible membrane 404 includes one or more markings 406 on a surface of the flexible membrane 404 that deform when the flexible membrane changes shape. The markings 406 may then be used to determine the pressure within the chamber 408 based on an amount of deformation in the markings or a pattern formed by the markings as the flexible membrane 404 changes shape. For example, the markings may include a plurality of straight lines when the flexible membrane 404 is flat, and which may deform into a pattern of curved lines according to an amount of curvature of the flexible membrane as it changes to a convex shape. In the examples of FIGS. 6 and 7 a plunger mechanism may be integrated with the flexible membrane, as previously described.

In some implementations, the flexible membrane may be fabricated to flex and deform responsive to the pressure caused by the fluid accumulated within the chamber. The membrane may be of a synthetic or semisynthetic material (e.g., polymer based material), and may have a degree of elasticity to deform. In some implementations, the membrane may be a thin plastic or PVC that snaps into either a flat, concaved, or convex form, depending on pressures within the chamber. The membrane may be a predetermined thickness and shape (e.g., a circle or ovoid).

In some implementations, the flexible membrane is configured to be substantially flat when a pressure within the chamber satisfies a first predetermined pressure threshold, and is configured to take on a convex shape responsive to the pressure satisfying a second predetermined pressure threshold. Accordingly, the flexible membrane may be configured to project away from the chamber responsive to the pressure becoming greater than the second predetermined pressure threshold, wherein the second predetermined pressure threshold is greater than or equal to the first predetermined pressure threshold.

According to various implementations, the chamber may be in the form of a basin with the input and output ports fluidically formed with sides of the basin and the flexible membrane covering the basin opening. The flexible membrane being able to change shape may include the flexible membrane being configured to switch from a default shape to an expanded shape responsive to the pressure satisfying a predetermined pressure threshold. In this regard, the chamber and flexible membrane may be configured to operate together to reduce the pressure within the chamber when switched to the expanded shape. For example, as the elasticity of the membrane allows the membrane to expand outward, the space created by the expansion is added to the internal volume of the chamber, thereby reducing the pressure. In some implementations, the membrane is designed to prevent return to the default shape. For example, if after expanding the pressure reduces, the membrane may not collapse (e.g., from the convex shape if plastic) or may become convex (e.g., in presence of negative pressure).

According to various aspects, the device functions as a pressure detection valve. When excessive pressure is introduced into an infusion line, the device, provides pressure detection by collecting fluid within the fluid chamber, which grows in volume due to expansion of the flexible membrane. The total volume of the chamber is determined by the chamber size constrained by shape of the flexible membrane. Accordingly, the pressure detection may be a function of the elasticity and total expansion of the membrane. Moreover, the device may be constructed in various sizes, depending on the expected volume of the expected pressurization bolus (e.g., from an associated infusion system). Similarly, the device may be fabricated with different pressure capacities to engage at different pressure. While, according to various implementations, the body may be fabricated by, for example, injection molded plastic, in some implementations, the body may be fabricated entirely out of a flexible membrane that expands when pressurized.

Advantages of the disclosed gas removal device include the ability to be molded and produced at high volumes, thereby driving down cost, while requiring little to no additional clinician training in the field. Furthermore, the device is configured such that it does not change priming volume (e.g., if length of tubing similar to length of device is replaced), and requires no external power source to operate.

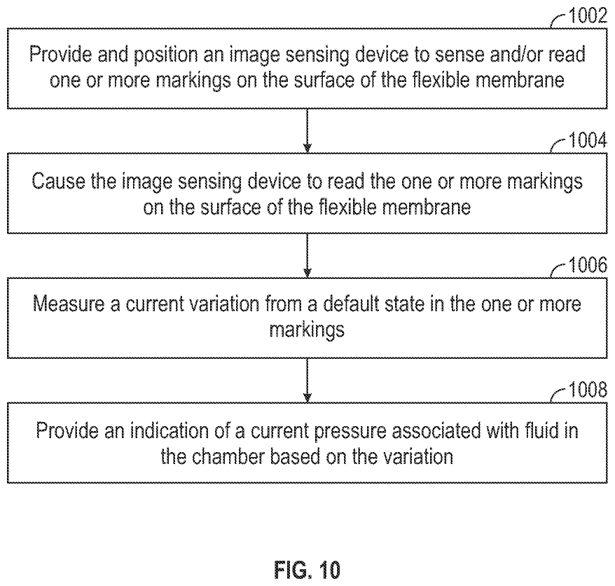

FIG. 10 depicts an example process for detecting a pressure fault with a pressure detection system, according to various aspects of the subject technology. For explanatory purposes, the various blocks of example process 1000 are described herein with reference to FIGS. 1-5, as well as the components and processes described herein. In some implementations, one or more of the blocks may be implemented apart from other blocks, and by one or more different processors or devices. Further, for explanatory purposes, the blocks of example process 1000 are described as occurring in serial, or linearly. However, multiple blocks of example process 1000 may occur in parallel. In addition, the blocks of example process 1000 need not be performed in the order shown and one or more of the blocks of example process 1000 need not be performed.

According to some implementations, an image sensing device 502 (including image sensing instrument 504) is provided in connection with the disclosed pressure relieve device 50 and positioned to sense and/or read the one or more markings 406 on the surface of the flexible membrane 404 (1002). A processor operating the image sensing device 502 may be configured to cause the sensing device 502 (e.g., by programming) to read the one or more markings on the surface of the flexible membrane 404 (1004). The processor operating the image sensing device 502 may be configured to then cause the sensing device 502 (e.g., by programming) to measure, based on the image sensing device reading the one or more markings, a current variation from a default state in the one or more markings (1006). The processor may further be configured to provide an indication of a current pressure associated with the fluid in the chamber based on the current variation in the markings from the default state (1008).

For example, the markings 406 may include a pattern of lines that curve as the pressure increases and the membrane 404 expands. The processor may be configured to detect and match a current pattern of curved lines with one or more predetermined patterns (e.g., stored in a memory such as a database), and to determine the current pressure based on indexing a matched pattern with a predetermined pressure value. In some implementations, the processor may be configured to determine an expansion state of the flexible membrane 404 based on the deformation of the markings 406 resulting from an amount of shape change of the flexible membrane 404, and to determine a deviation in the pressure within the chamber from a baseline pressure based on the determined expansion state. In some implementations, the processor may be configured to determine that a current variation in the marking(s) 406 corresponds to the current pressure in the chamber 408 satisfying a predetermined threshold pressure. In this regard, the indication provided may be a notification regarding the current pressure satisfying the predetermined pressure threshold.

In some implementations, the processor is communicatively connected to an infusion pump 10. In this regard, the processor may be configured to determine that an infusion pump 10 has initiated an infusion of the fluid. For example, the processor may be configured to determine that the infusion pump initiated the infusion based on the image sensing device 502 reading a predetermined variation from the default state in the markings. The processor may be configured to activate the image sensing device 502 to capture an image of the one or more markings on initiation of the infusion. The processor is then configured to compare the captured image to one or more predetermined patterns corresponding to a default expansion state and to determine, based on comparing the captured image to the one or more predetermined patterns, a threshold marking pattern for detecting an over pressure in the infusion line. The threshold marking pattern may be determined, at least in part, by indexing a threshold pressure value based on the default expansion state, and determining a marking pattern associated with an over pressure for the default expansion state. The processor is configured to then periodically monitor, with the image sensing device during the infusion, the one or more markings for the threshold marking pattern, and to provide an alert on detecting the threshold marking pattern.

In some implementations in which the processor is communicatively connected to an infusion pump 10 and determine when the infusion pump has initiated an infusion of the fluid, the processor is configured to determine that the current variation in the one or more markings corresponds to an over pressure associated with the infusion of the fluid, and to, responsive to determining that the current variation corresponds to an over pressure, (i) provide an alert indicating that the current pressure exceeded a safe pressure and (ii) signal the infusion pump to terminate the infusion. The signal may be a high/low binary signal, or may be in the form of a programmatic instruction communicated to the infusion device over a wireless 516 or wired interface 518 connection, or over a serial connection 520. The infusion device may then receive the signal and terminate the infusion responsive to the signal.

In some implementations, the processor is configured to determine that the infusion pump has initiated priming of an infusion line, and to, responsive to the pressure not satisfying the predetermined pressure threshold, providing an alert indicating that the priming of the infusion line is incomplete. In some implementations, the processor is configured to determine that the current variation in the one or more markings corresponds to the current pressure not satisfying a predetermined threshold pressure, and to provide a notification regarding the current pressure not satisfying the predetermined pressure threshold.

Many of the above-described example 1000, and related programming and configuring features, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

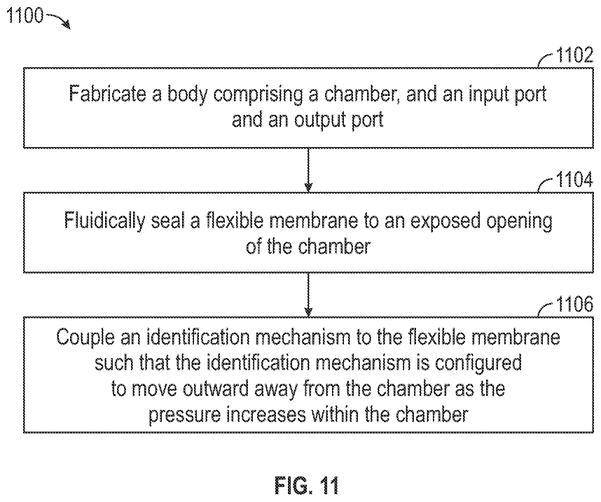

FIG. 11 depicts a second example process for fabricating or otherwise providing a pressure detection apparatus, according to various aspects of the subject technology. For explanatory purposes, the various blocks of example process 1100 are described herein with reference to FIGS. 1-8, as well as the components and processes described herein. In some implementations, one or more of the blocks may be implemented apart from other blocks, and by one or more different processors or devices. Further, for explanatory purposes, the blocks of example process 1100 are described as occurring in serial, or linearly. However, multiple blocks of example process 1100 may occur in parallel. In addition, the blocks of example process 1100 need not be performed in the order shown and one or more of the blocks of example process 1100 need not be performed.

In the depicted example, a body (e.g., body 302, 402, 602), including a chamber (e.g., chamber 303, 408 606), an input port (e.g., port 304, 410, 608) and an output port (e.g., port 306, 412, 610) is fabricated (1102). The chamber is configured to accumulate fluid from an upstream portion of an infusion line fluidly coupled to the input port, and to supply the fluid to a downstream portion of the infusion line fluidly connected to the output port. A flexible membrane 308, 404 is fluidically sealed to an exposed opening of the chamber such as to prevent the fluid from passing through the exposed opening (1104). According to various implementations, the flexible membrane is configured to change shape responsive to a pressure caused by the fluid accumulated within the chamber.

An identification mechanism is coupled to the flexible membrane and positioned to move outward, away from the chamber (1106), for example, as the pressure increases and the flexible membrane expands. In this regard, the distance that the identification mechanism travels responsive to the pressure increase is indicative of the pressure increase.

According to various implementations, the identification mechanism includes a plunger 614 coupled to the flexible membrane and positioned to project outward, away from the chamber. As described previously, a casing 616 encompassing at least a portion of the flexible membrane 604 may be coupled to at least a portion of the body. The casing may include an aperture 612 at a location of the plunger so that the plunger can pass through the aperture. In this regard, the plunger 614 moves with the flexible membrane so that, as the pressure increases and the flexible membrane expands, the plunger moves to extend further beyond the aperture and the casing.

In some implementations, with reference to FIG. 6E, the pressure detection apparatus 50 includes a taring mechanism 622 operatively coupled to the casing and mechanically movable in a lateral direction 624 along a length of the plunger 614 with respect to the casing such as to identify a location on the plunger 614 with a portion (e.g., a surface) of the taring mechanism. The taring mechanism 622 may be implemented as a circular dial that is threaded on a threaded collar 620 of the casing 616. The threaded collar may include the aperture 612. In this regard, the taring mechanism 622 may be mechanically movable by way of being turned 626 about the collar according to the threads of the threaded collar.

In some implementations, with reference to FIGS. 7A, 7B, and 7C, the pressure detection apparatus 50 may include a plunger housing 630 coupled to an outer portion of the casing 616 such as to enclose the plunger and the aperture. The housing 630 may include one or more openings 634 to expose a portion of the plunger enclosed inside the housing. In implementations in which the casing and/or body are circular, the housing may also be circular with a diameter substantially smaller than the diameter of the casing and/or body. In the example depicted in FIG. 7A, the diameter of the housing 630 is approximately half of the diameter of the casing 616.

The plunger may include a first identifier and a second identifier laterally disposed on the plunger, one of which may be visible to a user through the opening 634 at any one time depending on the pressure within the pressure detection apparatus 50. In some implementations, the identifiers are color coded. For example, the plunger 614 may be color coded green and red. The first identifier (e.g., green) is viewable through the opening 634 when the plunger is in a first position associated with a first pressure (e.g., a stable pressure), and the second identifier (e.g., green) is viewable through the opening 634 when the plunger 614 is in a second position associated with a second pressure (e.g., a high pressure fault condition).

In some implementations, plunger housing is adjustable to calibrate (e.g., zero out) the pressure reading. In this regard, the housing 630 may be operatively coupled to the casing by way of a threaded connection. In this manner, the housing 630 may be mechanically movable in a lateral direction along the length of the plunger with respect to the casing such as to reposition the opening. While the pressure is in an initial or default state, the housing may be adjusted in a lateral direction along the length of the plunger until the first identifier associated with the default state is visible through the opening.

In some implementations, as depicted in FIG. 8, the identification mechanism includes a plate coupled to the flexible membrane. The plate 802 may be positioned parallel to the chamber opening and fluidically sealed to chamber by way of one side of the flexible membrane being sealed to the perimeter of the inner side of an interior of the chamber, and the other side sealed to the plate. As the pressure increases in the chamber, the flexible membrane expands and/or stretches, and the plate moves unidirectionally away 804 from the body 602 and the chamber 608.

The body 602 may include a rectangular structure having at least one flat side. The side upon which the plate 802 rests when in a default state may also be flat. The example depicted in FIG. 8 has four flat sides. In some implementations, the pressure detection apparatus 50 includes one or more transparent panels removably coupled to the body. A transparent panel 806 may abut a flat side such as to be parallel with the flat side and perpendicular to the plate. In this regard, as shown in FIG. 8, movement of an edge of the plate from the body, and distance that the plate travels responsive to the pressure increase, is viewable through the transparent panel.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

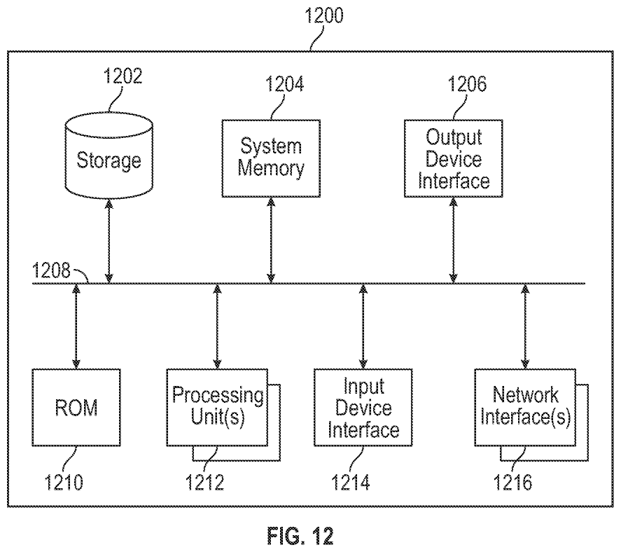

FIG. 12 is a conceptual diagram illustrating an example electronic system 1200 for facilitating pressure sensing in a pressure detection system, according to aspects of the subject technology. Electronic system 1200 may be a computing device for execution of software associated with one or more portions or steps of process 1200, or components and processes provided by FIGS. 1-11, including but not limited to computing device 8, processor 514, computing hardware within an infusion device 10, or an operably connected remote device (e.g., a mobile device). Electronic system 1200 may be representative, in combination with the disclosure regarding FIGS. 1-7. In this regard, electronic system 1200 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 1200 may include various types of computer readable media and interfaces for various other types of computer readable media. In the depicted example, electronic system 1200 includes a bus 1208, processing unit(s) 1212, a system memory 1204, a read-only memory (ROM) 1210, a permanent storage device 1202, an input device interface 1214, an output device interface 1206, and one or more network interfaces 1216. In some implementations, electronic system 1200 may include or be integrated with other computing devices or circuitry for operation of the various components and processes previously described.

Bus 1208 collectively represents all system, peripheral, and chipset buses that communicatively connect the numerous internal devices of electronic system 1200. For instance, bus 1208 communicatively connects processing unit(s) 1212 with ROM 1210, system memory 1204, and permanent storage device 1202.

From these various memory units, processing unit(s) 1212 retrieves instructions to execute and data to process, in order to execute the processes of the subject disclosure. The processing unit(s) can be a single processor or a multi-core processor in different implementations.

ROM 1210 stores static data and instructions that are needed by processing unit(s) 1212 and other modules of the electronic system. Permanent storage device 1202, on the other hand, is a read-and-write memory device. This device is a non-volatile memory unit that stores instructions and data even when electronic system 1200 is off. Some implementations of the subject disclosure use a mass-storage device (such as a magnetic or optical disk and its corresponding disk drive) as permanent storage device 1202.

Other implementations use a removable storage device (such as a floppy disk, flash drive, and its corresponding disk drive) as permanent storage device 1202. Like permanent storage device 1202, system memory 1204 is a read-and-write memory device. However, unlike storage device 1202, system memory 1204 is a volatile read-and-write memory, such as a random access memory. System memory 1204 stores some of the instructions and data that the processor needs at runtime. In some implementations, the processes of the subject disclosure are stored in system memory 1204, permanent storage device 1202, and/or ROM 1210. From these various memory units, processing unit(s) 1212 retrieves instructions to execute and data to process in order to execute the processes of some implementations.

Bus 1208 also connects to input and output device interfaces 1214 and 1206. Input device interface 1214 enables the user to communicate information and select commands to the electronic system. Input devices used with input device interface 1214 include, e.g., alphanumeric keyboards and pointing devices (also called “cursor control devices”). Output device interfaces 1206 enables, e.g., the display of images generated by the electronic system 1200. Output devices used with output device interface 1206 include, e.g., printers and display devices, such as cathode ray tubes (CRT) or liquid crystal displays (LCD). Some implementations include devices such as a touchscreen that functions as both input and output devices.

Also, as shown in FIG. 12, bus 1208 also couples electronic system 1200 to a network (not shown) through network interfaces 1216. Network interfaces 1216 may include, e.g., a wireless access point (e.g., Bluetooth or WiFi) or radio circuitry for connecting to a wireless access point. Network interfaces 1216 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1200 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented. Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A pressure detection system comprising: a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and a flexible membrane fluidically sealed to the exposed opening such as to prevent the fluid from passing through the exposed opening, and configured to change shape and expand responsive to an increase in pressure caused by the fluid accumulated within the chamber; a identification mechanism coupled to the flexible membrane and positioned to move outward, away from the chamber, as the pressure increases and the flexible membrane expands, wherein a distance that the identification mechanism travels responsive to the pressure increase is indicative of the pressure increase.

Clause 2. The pressure detection system of Clause 1, wherein the identification mechanism comprises: a plunger coupled to the flexible membrane and positioned to project outward, away from the chamber, the pressure detection system further comprising: a casing encompassing at least a portion of the flexible membrane and coupled to at least a portion of the body, the casing comprising an aperture at a location of the plunger, the plunger passing through the aperture, wherein the pressure detection system is configured such that, as the pressure increases and the flexible membrane expands, the plunger moves to extend further beyond the aperture and the casing.

Clause 3. The pressure detection system of Clause 2, further comprising: a taring mechanism operatively coupled to the casing and mechanically movable in a lateral direction along a length of the plunger with respect to the casing such as to identify a location on the plunger with a portion of the taring mechanism.

Clause 4. The pressure detection system of Clause 3, wherein the casing comprises a threaded collar and the threaded collar comprises the aperture, and wherein the taring mechanism is coupled to the casing by way of being threaded onto threads of the threaded collar, the taring mechanism mechanically movable by way of being turned about the threaded collar according to the threads of the threaded collar.

Clause 5. The pressure detection system of Clause 2, further comprising: a plunger housing coupled to an outer portion of the casing such as to encompass the plunger and the aperture, wherein the plunger includes a first identifier and a second identifier laterally disposed on the plunger; wherein the plunger housing comprises an opening at a location on the plunger housing to expose the first identifier when the plunger is in a first position associated with a first pressure, and to expose the second identifier when the plunger is in a second position associated with a second pressure.

Clause 6. The pressure detection system of Clause 5, wherein the plunger housing is operatively coupled to the casing and mechanically movable in a lateral direction along the length of the plunger with respect to the casing such as to reposition the opening.

Clause 7. The pressure detection system of Clause 1, wherein the identification mechanism comprises: a plate coupled to the flexible membrane and positioned parallel to the chamber, wherein the flexible membrane is fluidically sealed to an inner side of an interior of the chamber, wherein, as the pressure increases and the flexible membrane expands, the plate moves unidirectionally away from the body and the chamber.

Clause 8. The pressure detection system of Clause 7, wherein the body comprises a rectangular structure having at least one flat side, the pressure detection system further comprising: a transparent panel removably coupled to the body such as to be parallel to the at least one flat side and perpendicular to the plate, wherein movement of an edge of the plate from the body, and distance that the plate travels responsive to the pressure increase, is viewable through the transparent panel.

Clause 9. The pressure detection system of Clause 8, wherein the plate is rectangular and the flexible membrane comprises a four sided bellow with each of the four sides at least partially disposed within the chamber.

Clause 10. A method, comprising: providing a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and fluidically sealing a flexible membrane to the exposed opening such as to prevent the fluid from passing through the exposed opening, and configured to change shape and expand responsive to an increase in pressure caused by the fluid accumulated within the chamber; coupling an identification mechanism to the flexible membrane and positioned to move outward, away from the chamber, as the pressure increases and the flexible membrane expands, wherein a distance that the identification mechanism travels responsive to the pressure increase is indicative of the pressure increase.

Clause 11. A pressure detection system comprising: a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and a flexible membrane fluidically sealed to the exposed opening such as to prevent the fluid from passing through the exposed opening, and configured to change shape responsive to a pressure caused by the fluid accumulated within the chamber satisfying a predetermined threshold, the flexible membrane comprising one or more markings on a surface of the flexible membrane that deform when the flexible membrane changes shape.

Clause 12. The pressure detection system of Clause 11, further comprising: an image sensing device; and one or more processors configured to: cause the image sensing device to read the one or more markings on the surface of the flexible membrane; measure, based on the image sensing device reading the one or more markings, a current variation from a default state in the one or more markings; and provide an indication of a current pressure associated with the fluid in the chamber based on the current variation.

Clause 13. The pressure detection system of Clause 12, wherein the one or more processors is further configured to: determine that the current variation in the one or more markings corresponds to the current pressure satisfying a predetermined threshold pressure; and provide a notification regarding the current pressure satisfying the predetermined pressure threshold.

Clause 14. The pressure detection system of Clause 12, further comprising: an infusion pump, wherein the one or more processors is further configured to: determine that the infusion pump has initiated an infusion of the fluid; activate the image sensing device to capture an image of the one or more markings on initiation of the infusion; compare the captured image to one or more predetermined patterns corresponding to a default expansion state; determining, based on comparing the captured image to the one or more predetermined patterns, a threshold marking pattern for detecting an over pressure in the infusion line; periodically monitoring, with the image sensing device during the infusion, the one or more markings for the threshold marking pattern; and providing an alert on detecting the threshold marking pattern.

Clause 15. The pressure detection system of Clause 12, further comprising: an infusion pump, wherein the one or more processors is further configured to: determine that the infusion pump has initiated an infusion of the fluid; determining that the current variation in the one or more markings corresponds to an over pressure associated with the infusion of the fluid; and responsive to determining that the current variation corresponds to an over pressure, (i) providing an alert indicating that the current pressure exceeded a safe pressure and (ii) signal the infusion pump to terminate the infusion, wherein the infusion is terminated responsive to the signal.

Clause 16. The pressure detection system of Clause 15, wherein the one or more processors are configured to: determine that the infusion pump initiated the infusion of the fluid based on the image sensing device reading a first variation from the default state in the one or more markings.

Clause 17. The pressure detection system of Clause 12, wherein the one or more processors is further configured to: determine that the current variation in the one or more markings corresponds to the current pressure not satisfying a predetermined threshold pressure; and provide a notification regarding the current pressure not satisfying the predetermined pressure threshold.

Clause 18. The pressure detection system of Clause 15, further comprising: an infusion pump; wherein the one or more processors is further configured to: determine that the infusion pump has initiated priming of an infusion line; responsive to the pressure not satisfying the predetermined pressure threshold, providing an alert indicating that the priming of the infusion line is incomplete.

Clause 19. The pressure detection system of Clause 12, wherein the flexible membrane is configured to be flat when the current pressure satisfies a first predetermined pressure threshold, and is configured to take on a convex shape responsive to the current pressure satisfying a second predetermined pressure threshold, wherein the one or more markings comprise a plurality of straight lines when the flexible membrane is flat, and which deform into a pattern of curved lines according to an amount of curvature of the flexible membrane when in the convex shape, and wherein the one or more processors are configured to: detect and match the pattern of curved lines with one or more predetermined patterns; and determine the current pressure based on indexing a matched pattern with a predetermined pressure value.

Clause 20. The pressure detection system of Clause 12, wherein the one or more processors are further configured to: determine an expansion state of the flexible membrane based on the markings read from the surface of the flexible membrane, the expansion state comprising an amount of shape change; and determine a deviation in the pressure within the chamber from a baseline pressure based on the determined expansion state.

Clause 21. A method for providing pressure detection system comprising: providing a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and fluidically sealing a flexible membrane to an exposed opening of the chamber such as to prevent the fluid from passing through the exposed opening, and configured to change shape responsive to a pressure caused by the fluid accumulated within the chamber satisfying a predetermined threshold, the flexible membrane comprising one or more markings on a surface of the flexible membrane that deform when the flexible membrane changes shape.

Clause 22. The method of Clause 21, further comprising: configuring an image sensing device to read the one or more markings on the surface of the flexible membrane; configuring a processor to measure, based on the image sensing device reading the one or more markings, a current variation from a default state in the one or more markings; and configuring the processor to provide an indication of a current pressure associated with the fluid in the chamber based on the current variation.

Clause 23. The method Clause 22, further comprising: configuring the processor to determine that the current variation in the one or more markings corresponds to the current pressure satisfying a predetermined threshold pressure; and configuring the processor to provide a notification regarding the current pressure satisfying the predetermined pressure threshold.

Clause 24. The method of Clause 22, further comprising: configuring the processor to determine that an infusion pump has initiated an infusion of the fluid; configuring the processor to activate the image sensing device to capture an image of the one or more markings on initiation of the infusion; configuring the processor to compare the captured image to one or more predetermined patterns corresponding to a default expansion state; configuring the processor to determining, based on comparing the captured image to the one or more predetermined patterns, a threshold marking pattern for detecting an over pressure in the infusion line; configuring the processor to periodically monitoring, with the image sensing device during the infusion, the one or more markings for the threshold marking pattern; and configuring the processor to providing an alert on detecting the threshold marking pattern.

Clause 25. The method of Clause 22, further comprising: configuring the processor to determine that an infusion pump has initiated an infusion of the fluid; configuring the processor to determine that the current variation in the one or more markings corresponds to an over pressure associated with the infusion of the fluid; and configuring the processor to, responsive to determining that the current variation corresponds to an over pressure, (i) provide an alert indicating that the current pressure exceeded a safe pressure and (ii) signal the infusion pump to terminate the infusion, wherein the infusion is terminated responsive to the signal.

Clause 26. The method of Clause 25, further comprising: configuring the processor to determine that the infusion pump initiated the infusion of the fluid based on the image sensing device reading a first variation from the default state in the one or more markings.

Clause 27. The method of Clause 22, further comprising: configuring the processor to determine that the current variation in the one or more markings corresponds to the current pressure not satisfying a predetermined threshold pressure; and configuring the processor to provide a notification regarding the current pressure not satisfying the predetermined pressure threshold.

Clause 28. The method of Clause 25, further comprising: configuring the processor to determine that the infusion pump has initiated priming of an infusion line; configuring the processor to, responsive to the pressure not satisfying the predetermined pressure threshold, providing an alert indicating that the priming of the infusion line is incomplete.

Clause 29. The method of Clause 22, wherein the flexible membrane is configured to be flat when the current pressure satisfies a first predetermined pressure threshold, and is configured to take on a convex shape responsive to the current pressure satisfying a second predetermined pressure threshold, wherein the one or more markings comprise a plurality of straight lines when the flexible membrane is flat, and which deform into a pattern of curved lines according to an amount of curvature of the flexible membrane when in the convex shape, and wherein the process further comprises: configuring the processor to detect and match the pattern of curved lines with one or more predetermined patterns; and configuring the processor to determine the current pressure based on indexing a matched pattern with a predetermined pressure value.

Clause 30. The method of Clause 22, further comprising: configuring the processor to determine an expansion state of the flexible membrane based on the markings read from the surface of the flexible membrane, the expansion state comprising an amount of shape change configuring the processor to determine a deviation in the pressure within the chamber from a baseline pressure based on the determined expansion state.

Clause 31. A pressure relief system comprising: a body comprising a chamber, an input port and an output port, the chamber configured to accumulate fluid from an upstream portion of an infusion line fluidly coupled to the input port, and to supply the fluid to a downstream portion of the infusion line fluidly connected to the output port; and a flexible membrane fluidically sealed to an exposed opening of the chamber such as to prevent the fluid from passing through the exposed opening, wherein the flexible membrane is configured to change shape responsive to a pressure caused by the fluid accumulated within the chamber.

Clause 32. The pressure relief system of Clause 31, wherein the flexible membrane is of a predetermined thickness and shape configured to flex and deform responsive to the pressure caused by the fluid accumulated within the chamber.

Clause 33. The pressure relief system of Clause 32, wherein the flexible membrane is configured to be flat when a pressure within the chamber satisfies a first predetermined pressure threshold, and is configured to take on a convex shape responsive to the pressure satisfying a second predetermined pressure threshold.

Clause 34. The pressure relief system of Clause 33, wherein the flexible membrane is configured to project away from chamber responsive to the pressure becoming greater than the second predetermined pressure threshold, wherein the second predetermined pressure threshold is greater than or equal to the first predetermined pressure threshold.

Clause 35. The pressure relief system of Clause 31, wherein the chamber comprises a basin with the input and output ports fluidically formed with sides of the basin and the flexible membrane covering the basin opening.

Clause 36. The pressure relief system of Clause 31, wherein the flexible membrane being configured to change shape comprises the flexible membrane being configured to switch from a default shape to an expanded shape responsive to the pressure satisfying a predetermined pressure threshold.

Clause 37. The pressure relief system of Clause 36, wherein the chamber and flexible membrane are configured to operate together to reduce the pressure within the chamber when switched to the expanded shape.

Clause 38. The pressure relief system of Clause 36, wherein the flexible membrane is prevented from returning to the default shape after being switching to the expanded shape.

Clause 39. A process for forming a pressure relief apparatus, comprising: providing a body comprising a chamber, an input port and an output port, the chamber configured to accumulate fluid from an upstream tubing fluidly coupled to the input port, and to supply the fluid to a downstream tubing fluidly connected to the output port; fluidically sealing a flexible membrane to an exposed opening of the chamber such as to prevent the fluid from passing through the exposed opening, wherein the flexible membrane is configured and sealed such as to change shape responsive to a pressure caused by the fluid accumulated within the chamber.

Clause 40. The process of Clause 39, wherein the flexible membrane is of a predetermined thickness and shape configured to flex and deform responsive to the pressure caused by the fluid accumulated within the chamber.

Clause 41. The process of Clause 40, wherein the flexible membrane is configured to be substantially flat when a pressure within the chamber satisfies a first predetermined pressure threshold, and is configured to take on a convex shape responsive to the pressure satisfying a second predetermined pressure threshold.

Clause 42. The process of Clause 41, wherein the flexible membrane is configured to project away from the chamber responsive to the pressure becoming greater than the second predetermined pressure threshold, wherein the second predetermined pressure threshold is greater than or equal to the first predetermined pressure threshold.

Clause 43. The process of Clause 39, wherein the chamber comprises a basin with the input and output ports fluidically formed with sides of the basin and the flexible membrane covering the basin opening.

Clause 44. The process of Clause 39, wherein the flexible membrane being configured to change shape comprises the flexible membrane being configured to switch from a default shape to an expanded shape responsive to the pressure satisfying a predetermined pressure threshold.

Clause 45. The process of Clause 44, wherein the chamber and flexible membrane are configured to operate together to reduce the pressure within the chamber when switched to the expanded shape.

Clause 46. The process of claim Clause 44, wherein the flexible membrane is prevented from returning to the default shape after being switching to the expanded shape.

Clause 47. A pressure relief apparatus, comprising: a body comprising a chamber, an input port and an output port, the chamber configured to accumulate fluid from an upstream tubing fluidly coupled to the input port, and to supply the fluid to a downstream tubing fluidly connected to the output port; a flexible membrane fluidically sealed to an exposed opening of the chamber such as to prevent the fluid from passing through the exposed opening, wherein the flexible membrane is configured and sealed such as to change shape responsive to a pressure caused by the fluid accumulated within the chamber.

Clause 48. The pressure relief apparatus of Clause 47, wherein the flexible membrane is of a predetermined thickness and shape configured to flex and deform responsive to the pressure caused by the fluid accumulated within the chamber, and configured to be substantially flat in a default state while a pressure within the chamber satisfies a first predetermined pressure threshold, and to switch to an expanded state and take on a convex shape responsive to the pressure satisfying a second predetermined pressure threshold, wherein the flexible membrane is configured to project away from the chamber responsive to the pressure becoming greater than the second predetermined pressure threshold, wherein the second predetermined pressure threshold is greater than or equal to the first predetermined pressure threshold.

Clause 49. The pressure relief apparatus of Clause 48, wherein the chamber and flexible membrane are configured to operate together to reduce the pressure within the chamber when switched to the expanded state.

Clause 50. The pressure relief apparatus of Clause 48, wherein the flexible membrane is prevented from returning to the default shape after being switching to the expanded shape.

Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A pressure detection system comprising:
a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and
a flexible membrane fluidically sealed to the exposed opening such as to prevent the fluid from passing through the exposed opening, and configured to change shape and expand responsive to an increase in pressure caused by the fluid accumulated within the chamber;
a identification mechanism coupled to the flexible membrane and positioned to move outward, away from the chamber, as the pressure increases and the flexible membrane expands,
wherein a distance that the identification mechanism travels responsive to the pressure increase is indicative of the pressure increase.

2. The pressure detection system of claim 1, wherein the identification mechanism comprises:
a plunger coupled to the flexible membrane and positioned to project outward, away from the chamber,
the pressure detection system further comprising:
a casing encompassing at least a portion of the flexible membrane and coupled to at least a portion of the body, the casing comprising an aperture at a location of the plunger, the plunger passing through the aperture,
wherein the pressure detection system is configured such that, as the pressure increases and the flexible membrane expands, the plunger moves to extend further beyond the aperture and the casing.

3. The pressure detection system of claim 2, further comprising:
a taring mechanism operatively coupled to the casing and mechanically movable in a lateral direction along a length of the plunger with respect to the casing such as to identify a location on the plunger with a portion of the taring mechanism.

4. The pressure detection system of claim 3, wherein the casing comprises a threaded collar and the threaded collar comprises the aperture, and wherein the taring mechanism is coupled to the casing by way of being threaded onto threads of the threaded collar, the taring mechanism mechanically movable by way of being turned about the threaded collar according to the threads of the threaded collar.

5. The pressure detection system of claim 2, further comprising:
a plunger housing coupled to an outer portion of the casing such as to encompass the plunger and the aperture,
wherein the plunger includes a first identifier and a second identifier laterally disposed on the plunger;
wherein the plunger housing comprises an opening at a location on the plunger housing to expose the first identifier when the plunger is in a first position associated with a first pressure, and to expose the second identifier when the plunger is in a second position associated with a second pressure.

6. The pressure detection system of claim 5, wherein the plunger housing is operatively coupled to the casing and mechanically movable in a lateral direction along the length of the plunger with respect to the casing such as to reposition the opening.

7. The pressure detection system of claim 1, wherein the identification mechanism comprises:
a plate coupled to the flexible membrane and positioned parallel to the chamber, wherein the flexible membrane is fluidically sealed to an inner side of an interior of the chamber, wherein, as the pressure increases and the flexible membrane expands, the plate moves unidirectionally away from the body and the chamber.

8. The pressure detection system of claim 7, wherein the body comprises a rectangular structure having at least one flat side, the pressure detection system further comprising:
a transparent panel removably coupled to the body such as to be parallel to the at least one flat side and perpendicular to the plate,
wherein movement of an edge of the plate from the body, and distance that the plate travels responsive to the pressure increase, is viewable through the transparent panel.

9. The pressure detection system of claim 8, wherein the plate is rectangular and the flexible membrane comprises a four sided bellow with each of the four sides at least partially disposed within the chamber.

10. A method, comprising:
providing a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and
fluidically sealing a flexible membrane to the exposed opening such as to prevent the fluid from passing through the exposed opening, and configured to change shape and expand responsive to an increase in pressure caused by the fluid accumulated within the chamber;
coupling an identification mechanism to the flexible membrane and positioned to move outward, away from the chamber, as the pressure increases and the flexible membrane expands,
wherein a distance that the identification mechanism travels responsive to the pressure increase is indicative of the pressure increase.

11. A pressure detection system comprising:
a body comprising a chamber, an input port and an output port, the chamber having an exposed opening through a side of the body and being configured to accumulate fluid from an upstream portion of an infusion line fluidly coupled to the input port, and to supply the fluid to a downstream portion of the infusion line fluidly connected to the output port; and
a flexible membrane fluidically sealed to the exposed opening such as to prevent the fluid from passing through the exposed opening, and configured to change shape responsive to a pressure caused by the fluid accumulated within the chamber satisfying a predetermined threshold, the flexible membrane comprising one or more markings on a surface of the flexible membrane that deform when the flexible membrane changes shape.

12. The pressure detection system of claim 11, further comprising:
an image sensing device; and
one or more processors configured to:
cause the image sensing device to read the one or more markings on the surface of the flexible membrane;
measure, based on the image sensing device reading the one or more markings, a current variation from a default state in the one or more markings; and
provide an indication of a current pressure associated with the fluid in the chamber based on the current variation.

13. The pressure detection system of claim 12, wherein the one or more processors is further configured to:
determine that the current variation in the one or more markings corresponds to the current pressure satisfying a predetermined threshold pressure; and
provide a notification regarding the current pressure satisfying the predetermined pressure threshold.

14. The pressure detection system of claim 12, further comprising:
an infusion pump,
wherein the one or more processors is further configured to:
determine that the infusion pump has initiated an infusion of the fluid;
activate the image sensing device to capture an image of the one or more markings on initiation of the infusion;
compare the captured image to one or more predetermined patterns corresponding to a default expansion state;
determining, based on comparing the captured image to the one or more predetermined patterns, a threshold marking pattern for detecting an over pressure in the infusion line;
periodically monitoring, with the image sensing device during the infusion, the one or more markings for the threshold marking pattern; and
providing an alert on detecting the threshold marking pattern.

15. The pressure detection system of claim 12, further comprising:
an infusion pump,
wherein the one or more processors is further configured to:
determine that the infusion pump has initiated an infusion of the fluid;
determining that the current variation in the one or more markings corresponds to an over pressure associated with the infusion of the fluid; and
responsive to determining that the current variation corresponds to an over pressure, (i) providing an alert indicating that the current pressure exceeded a safe pressure and (ii) signal the infusion pump to terminate the infusion,
wherein the infusion is terminated responsive to the signal.

16. The pressure detection system of claim 15, wherein the one or more processors are configured to:
determine that the infusion pump initiated the infusion of the fluid based on the image sensing device reading a first variation from the default state in the one or more markings.

17. The pressure detection system of claim 12, wherein the one or more processors is further configured to:
determine that the current variation in the one or more markings corresponds to the current pressure not satisfying a predetermined threshold pressure; and
provide a notification regarding the current pressure not satisfying the predetermined pressure threshold.

18. The pressure detection system of claim 15, further comprising:
an infusion pump;
wherein the one or more processors is further configured to:
determine that the infusion pump has initiated priming of an infusion line;
responsive to the pressure not satisfying the predetermined pressure threshold, providing an alert indicating that the priming of the infusion line is incomplete.

19. The pressure detection system of claim 12, wherein the flexible membrane is configured to be flat when the current pressure satisfies a first predetermined pressure threshold, and is configured to take on a convex shape responsive to the current pressure satisfying a second predetermined pressure threshold, wherein the one or more markings comprise a plurality of straight lines when the flexible membrane is flat, and which deform into a pattern of curved lines according to an amount of curvature of the flexible membrane when in the convex shape, and
wherein the one or more processors are configured to:
detect and match the pattern of curved lines with one or more predetermined patterns; and
determine the current pressure based on indexing a matched pattern with a predetermined pressure value.

20. The pressure detection system of claim 12, wherein the one or more processors are further configured to:
determine an expansion state of the flexible membrane based on the markings read from the surface of the flexible membrane, the expansion state comprising an amount of shape change; and
determine a deviation in the pressure within the chamber from a baseline pressure based on the determined expansion state.
